(19) United States
(12) Patent Application Publication
(10) Pub. No.: US 2026/0284415 A1
(43) Pub. Date: Sep. 24, 2026

(54) MEDICAL DEVICE RETENTION FEATURE

(71) Applicant: Medtronic, Inc., Minneapolis, MN (US)

(72) Inventors: Brian P. Colin, Anoka, MN (US); Justin B. Seeley, Spring Park, MN (US)

(21) Appl. No.: 19/473,559

(22) PCT Filed: Mar. 20, 2024
(86) PCT No.: PCT/IB2024/052682

§ 371 Date: Oct. 8, 2025

Related U.S. Application Data
(60) Provisional application No. 63/496,220, filed on Apr. 14, 2023.

(51) Int. Cl. A61N 1/372; A61N 1/375

(52) U.S. Cl. CPC A61N 1/37205; A61N 1/37518; A61N 1/3756

(57) ABSTRACT

A system comprising: a delivery catheter comprising: an elongated body; a receptacle extending distally from a distal end of the elongated body along a longitudinal axis, the receptable defining an inner volume configured to retain an implantable medical device, wherein the inner volume is in fluid communication with a catheter lumen; and a locking collar disposed at the distal end of the elongated body, the locking collar comprising a first locking element; and a tether assembly configured to be disposed within the catheter lumen, wherein the tether assembly comprises: a tether head assembly configured to receive an attachment member of the implantable medical device; and a second locking element disposed on an outer surface of the tether head assembly, wherein the second locking element is configured to interface with the first locking element of the locking collar to inhibit unintended distal movement of the implantable medical device within the receptacle. Corresponding tether head assembly comprising an inner retainer coupled and extending distally from a distal end of an elongated member; an outer retainer defining a channel configured to receive the inner retainer, wherein the inner retainer and the outer retainer define a receptacle configured to receive an attachment member of a medical device; a sheath configured to receive the inner retainer and the outer retainer; a first locking element disposed on an outer surface of the sheath.

This application claims the benefit of U.S. Provisional Patent Application Ser. No. 63/496,220, filed Apr. 14, 2023, the entire content of which is incorporated herein by reference.

### TECHNICAL FIELD

This disclosure relates generally to medical devices, and, more particularly, to systems for delivering medical devices.

### BACKGROUND

Some types of implantable medical devices (IMDs), such as cardiac pacemakers or implantable cardioverter defibrillators systems, may be used to provide cardiac sensing and therapy for a patient via one or more electrodes. Some IMDs include an implantable pulse generator that includes a housing that encloses electronic components, which may be configured to be implanted subcutaneously in the chest of the patient or within a chamber of a heart of the patient, as examples. IMDs having a pulse generator that is configured to be implanted within a chamber of the heart may be referred to as an intracardiac device or a leadless implantable medical device. A medical device delivery system including a delivery catheter may be used to deliver an intracardiac device transvenously to an implant site within a heart of a patient and release the device after the device has been fixed at the implant site. The medical device delivery system then may be withdrawn from the patient.

### SUMMARY

In general, this disclosure is directed to devices, systems, and techniques for retention of a medical device within a medical device delivery system. The medical device may be connected to a tether assembly of the medical device delivery system and may be disposed within a catheter lumen of a delivery catheter of the medical device delivery system. The medical device and the tether assembly may be advanced out of a distal end of the delivery catheter, retracted proximally into the catheter lumen of the delivery catheter, and/or rotated within the catheter lumen of the delivery catheter via a force transmitted along an elongated member coupled to the tether assembly. The delivery catheter may include locking element(s) that interface with locking element(s) on the tether assembly to inhibit unintended movement, e.g., longitudinal movement and/or rotation of the tether assembly relative to the delivery catheter.

Other medical device delivery systems may include a tether assembly and medical device that is freely movable relative to the delivery catheter. In such examples, a clinician may retain the medical device and the tether assembly within the catheter lumen of the delivery catheter by coupling a proximal end of the elongated member to the delivery catheter. Once the delivery catheter is at a target implant site, the clinician may uncouple the elongated member from the delivery catheter and advance the medical device out of the delivery catheter. In some examples, a clinician may inject a fluid (e.g., a contrast fluid) into a blood vessel of a patient via the catheter lumen of the delivery catheter, e.g., to aid in the visualization of the delivery catheter within the blood vessel. During injection, the fluid may apply a force on the medical device and/or the tether assembly and may cause unintentional advancement of the medical device and/or the tether assembly distally out of the catheter lumen of the delivery catheter. Once the medical device and/or the tether assembly is advanced out of the delivery catheter, the medical device may interact with the tissue of the patient and lead to unintended effects such as damage to the medical device and/or implantation of the medical device outside of the target implant site. In some examples, during navigation of the delivery catheter within vasculature of the patient, the tortuosity of the vasculature may cause deflection of the delivery catheter and a reduction in the overall length of the delivery catheter due to the deflection. The reduction in the overall length of the delivery catheter may cause medical device and/or the tether assembly to protrude from the delivery catheter and may lead to the unintended effects described above.

This disclosure describes devices, systems, and methods for coupling the tether assembly to a locking collar of the delivery catheter to inhibit unintended distal movement of the medical device and the tether assembly relative to the delivery catheter. The locking collar may be disposed proximal to a receptacle on the delivery catheter that is configured to retain the medical device. Each of the locking collar and the tether assembly may include a locking element. The locking element may be configured to interface with one another to inhibit distal movement of the tether assembly. The locking elements may include locking protrusions and/or locking recesses disposed on an inner surface of the locking collar and/or on an outer surface of the tether assembly. The tether assembly may be rotated about a longitudinal axis and relative to the delivery catheter to lock or unlock the tether assembly from the delivery catheter.

Example medical device delivery systems described herein may provide several benefits over other medical device delivery systems. The retention features of the example medical device delivery systems may reduce a risk of an unintended protrusion of the medical device and/or the tether assembly from the delivery catheter and may reduce an amount of time required to implant the medical device by ensuring that the delivery catheter delivers the medical device to the target implant site without damage to the medical device. In some examples, the retention features of the example medical device delivery system may eliminate the risk of accidental protrusion of the medical device from the delivery catheter due to deflection of the delivery catheter and may allow the use of a more flexible delivery catheter design and/or more flexible materials for a delivery catheter of a medical device delivery system. A more flexible delivery catheter may allow for a greater range of use of the medical device delivery system (e.g., to areas in a patient with more tortuous vasculature).

In some examples, this disclosure describes a system comprising: a delivery catheter comprising: an elongated body extending from a proximal end to a distal end along a longitudinal axis, wherein the elongated body defines a catheter lumen extending from the proximal end to the distal end; a receptacle extending distally from the distal end of the elongated body along the longitudinal axis, the receptable defining an inner volume configured to retain an implantable medical device, wherein the inner volume is in fluid communication with the catheter lumen; and a locking collar disposed at the distal end of the elongated body, the locking collar comprising a first locking element; and a tether assembly configured to be disposed within the catheter lumen, wherein the tether assembly comprises: a tether head assembly configured to receive an attachment member of the implantable medical device; and second locking element disposed on an outer surface of the tether head assembly, wherein the second locking element is configured to interface with the first locking element of the locking collar to inhibit unintended distal movement of the implantable medical device within the receptacle.

In some examples, this disclosure describes A tether head assembly comprising: an inner retainer coupled and extending distally from a distal end of an elongated member; an outer retainer defining a channel configured to receive the inner retainer, wherein the inner retainer and the outer retainer define a receptacle configured to receive an attachment member of a medical device; a sheath configured to receive the inner retainer and the outer retainer; and a first locking element disposed on an outer surface of the sheath, wherein the tether head assembly is configured to be disposed within a catheter lumen of an elongated body of a delivery catheter, wherein the elongated member is configured to transmit a force from a proximal end of the elongated member to the inner retainer to move the tether head assembly within the catheter lumen of the delivery catheter. and wherein the first locking element is configured to interface with a second locking element disposed within an inner lumen to inhibit unintended movement of the tether head assembly towards a distal end of the elongated body of the delivery catheter.

In some examples, this disclosure describes a catheter comprising: an elongated body extending from a proximal end to a distal end along a longitudinal axis, wherein the elongated body defines a catheter lumen extending from the proximal end to the distal end; a receptacle extending distally from the distal end of the elongated body along the longitudinal axis, the receptable defining an inner volume configured to retain an implantable medical device, wherein the inner volume is in fluid communication with the catheter lumen; and a locking collar disposed at the distal end of the elongated body, the locking collar comprising a first locking element, wherein the first locking element is configured to interface with a second locking element on an outer surface of a tether head assembly disposed within the catheter lumen and attached to the implantable medical device to prevent unintended distal movement of the implantable medical device along the longitudinal axis.

In some examples, this disclosure describes a method comprising: advancing a tether head assembly of a tether assembly within a catheter lumen of an elongated body of a catheter to a locking collar disposed within a distal portion of the catheter via an elongated member coupled to a proximal end of the tether head assembly, wherein an inner surface of the locking collar defines a locking collar lumen in fluid communication with the catheter lumen; rotating the tether assembly about a longitudinal axis of the catheter from an unlocked orientation to a locked orientation to cause a first locking element of the locking collar to interface with a second locking element on the tether head assembly and inhibit unintended distal movement of the tether head assembly along the longitudinal axis; placing a medical device in an inner volume of a receptacle of the catheter, wherein the receptacle is affixed to the distal end of the elongated body; and attaching an attachment member of the medical device to the tether head assembly.

In some examples, this disclosure describes a method comprising: advancing a catheter through vasculature of a patient to a target tissue site, wherein the catheter comprises: an elongated body defining a catheter lumen, and a locking collar disposed within a distal portion of the catheter, the locking collar defining a first locking element, and a receptacle distal to the locking collar, wherein a tether head assembly of a tether assembly is disposed within the catheter lumen in a locked orientation, wherein the tether head assembly is coupled to an attachment member of a medical device, wherein the medical device is disposed within the receptacle without protruding from a distal end of the receptacle, and wherein a second locking element on an outer surface of the tether head assembly interfaces with the first locking element of the locking collar to inhibit unintended distal movement of the medical device; rotating the tether head assembly about a longitudinal axis of the catheter from the locked orientation to an unlocked orientation, wherein the second locking element does not interface with the first locking element when the tether head assembly is in the unlocked orientation; implanting the medical device within tissue at the target tissue site; and releasing the attachment member of the medical device from the tether head assembly.

This summary is intended to provide an overview of the subject matter described in this disclosure. It is not intended to provide an exclusive or exhaustive explanation of the apparatus and methods described in detail within the accompanying drawings and description below. Further details of one or more examples are set forth in the accompanying drawings and the description below.

### BRIEF DESCRIPTION OF THE DRAWINGS

The details of one or more examples of this disclosure are set forth in the accompanying drawings and the descriptions below. Other features, objects, and advantages of this disclosure will be apparent from the description and drawings, and from the claims.

FIG. 1 is a conceptual diagram illustrating an example medical device implanted in the heart of a patient, in accordance with one or more aspects of this disclosure.

FIG. 2 is a plan drawing illustrating an example medical device delivery system for delivering an IMD to a location within a heart.

FIG. 3 is a conceptual drawing illustrating, in conjunction with tissue of a heart, a distal portion of the example medical device delivery system of FIG. 2 carrying an example medical device.

FIG. 4 is a perspective diagram illustrating an example tether head assembly and locking collar of the example medical device delivery system of FIG. 3 in a locked orientation.

FIG. 5 is a perspective diagram illustrating the example tether head assembly and locking collar of FIG. 4 in an unlocked orientation.

FIG. 6. is a perspective diagram illustrating a front view of the example tether head assembly and locking collar of FIG. 4 in the unlocked orientation.

FIG. 7 is a cross-sectional view of the highlighted portion B of FIG. 3 including the tether assembly, locking collar, and a proximal portion of the medical device, where the cross-section is taken along line A-A of FIG. 3 in a plane parallel to a longitudinal axis of the tether assembly and a longitudinal axis of the medical device.

FIG. 8 is a flowchart illustrating an example process of implanting a medical device with the medical device delivery system of FIG. 2.

### DETAILED DESCRIPTION

In general, this disclosure describes example medical device delivery systems. Such medical device delivery systems may include a delivery catheter and a tether assembly disposed within a catheter lumen of the delivery catheter. The tether assembly is configured to releasably retain an attachment member of a medical device (e.g., an intracardiac device). Although the example tether assemblies are generally described herein as being configured for delivering an implantable medical device (IMD), it should be understood that any of the example tether assemblies described herein alternatively may be configured for delivering other types of medical devices.

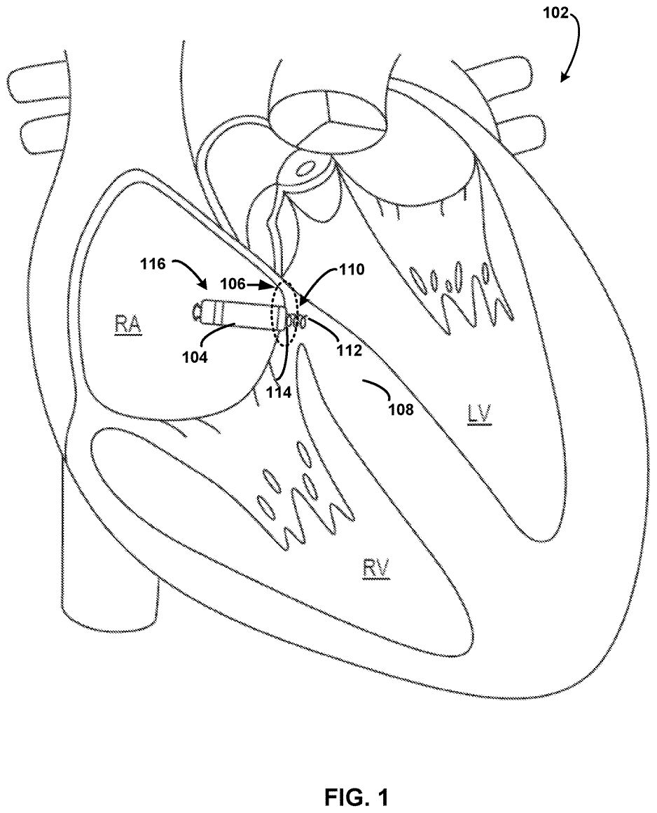

FIG. 1 is a conceptual diagram illustrating an example device 104 implanted in the heart 102 of a patient, in accordance with one or more aspects of this disclosure. Device 104 is shown implanted in the right atrium (RA) of the patient's heart 102 in a target implant region 106, such as the triangle of Koch, in heart 102 of the patient with a distal end of device 104 directed toward the left ventricle (LV) of the patient's heart 102. Although in the example of FIG. 1 the distal end of device 104 is directed toward the LV, the distal end may be directed to other targets, such as interventricular septum of heart 102. Target implant region 106 may lie between the bundle of His and the coronary sinus and may be adjacent the tricuspid valve.

Device 104 includes a distal end 110 and a proximal end 116. Distal end 110 includes a first electrode 112, and a second electrode 114. First electrode 112 may define a helical shape, e.g., as illustrated in FIG. 1. First electrode 112 extends from distal end 110 and may penetrate through the wall tissue of a first chamber (e.g., the RA in the illustrated example) into wall tissue of a second chamber (e.g., ventricular myocardium 108 of the LV in the illustrated example). Second electrode 114 may be disposed on a ramp extending distally from distal end 110 and is configured to be placed in contact with the wall tissue of the first chamber without penetration of the wall tissue of the first chamber by second electrode 114. Second electrode 114 may contact the wall tissue of the first chamber as first electrode 112 penetrates the wall tissue of the first chamber.

The configuration of electrodes 112 and 114 illustrated in FIG. 1 allows device 104 to sense cardiac signals and/or deliver cardiac pacing to multiple chambers of heart 102, e.g., the RA and ventricle(s) in the illustrated example. In this manner, the configuration of electrodes 112 and 114 may facilitate the delivery of A-V synchronous pacing by single device 104 implanted within the single chamber, e.g., the RA. While device 104 is implanted at target implant region 106 to sense in and/or pace the RA and ventricle(s) in the example shown in FIG. 1, a device having an electrode configuration in accordance with the examples of this disclosure may be implanted at any of a variety of locations to sense in and/or pace any one, two or more chambers of heart 102. For example, device 104 may be implanted at region 106 or another region, and first electrode 112 may extend into tissue, e.g., myocardial tissue, of the LV or interventricular septum to, for example, facilitate the delivery of A-V synchronous pacing. Furthermore, a device having an electrode configuration in accordance with the examples of this disclosure may be implanted at any of a variety of locations within a patient for sensing and/or delivery of therapy to other patient tissue. In some examples, first electrode 112 extends into the tissue of heart 102 at region 106 and affix device 104 to the tissue of heart 102.

FIG. 1 illustrates one example of device 104. Other examples of medical devices include, but are not limited to, implantable medical device (e.g., an implantable stimulators, implantable leads) configured to be fixed at any location or tissue of the body.

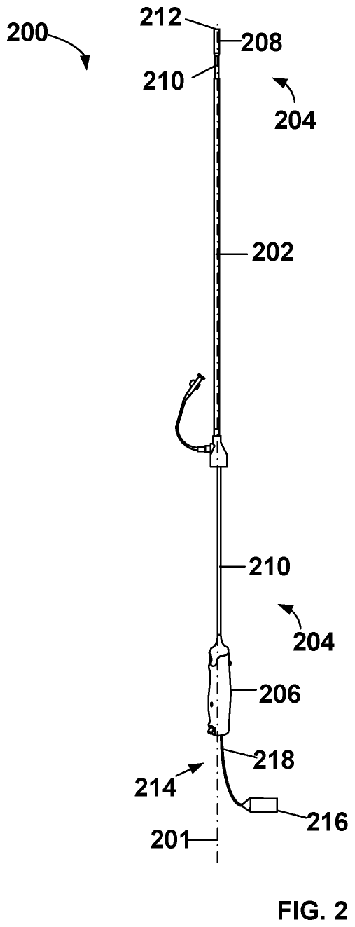

FIG. 2 is a plan drawing illustrating an example medical device delivery system 200 for delivering a medical device (, e.g., device 104) to a location within heart 102. Although described herein in the context of delivering a medical device into the vasculature, e.g., to heart 102, the devices, systems, and techniques of this disclosure may be used to deliver a medical device to any anatomical location on the patient.

System 4 includes an introducer 202, a delivery catheter 204, and a tether assembly 214. Introducer 202 is an elongated member defining an interior lumen. Introducer 202 is configured to be inserted, such as by a physician, into a vasculature of a patient to provide a rigid channel, via the interior lumen, through which to insert a medical instrument, a device, or other therapy.

Delivery catheter 204 is configured to be inserted through the lumen of introducer 202 to deliver device 104 within the vasculature. Delivery catheter 204 includes an elongated body 210, a handle 206 and a receptacle 208. Handle 206 is disposed at a proximal end of elongated body 210, and may include one or more elements (such as buttons, switches, etc.) configured to control the motion of the distal end of elongated body 210 and release of device 104 from receptacle 208, as examples.

Receptacle 208 is disposed at a distal end of elongated body 210. Receptacle 208 includes a hollow cylindrical body configured to house and support device 104 while device 104 is being implanted within a vasculature of a patient. For example, a clinician may insert the distal end of delivery catheter 204, including receptacle 208, through the lumen of introducer 202, which is disposed within a vasculature of a patient. Once receptacle 208 has extended through the distal end of introducer 202 and reached an implant site within the patient, the physician may release device 104 from a distal opening 212 of receptacle 208 and withdraw delivery catheter 204 proximally through introducer 202.

Tether assembly 214 extends through a catheter lumen defined delivery catheter, e.g., including handle 206 and elongated body 210. Tether assembly 214 includes an elongated member 218, a tether handle assembly 216 at a proximal end of elongated member 218, and a tether head assembly (not pictured) at a distal end of elongated member 218. The elongated member 218 may be formed from a metallic alloy. In some examples, at least portions of elongated member 218 may be a hypotube.

Tether assembly 214 may be of sufficient length that a clinician may manipulate tether handle assembly 216 to advance the tether head assembly out of distal opening 212 of receptacle 208. In some examples, with the tether head assembly outside of receptacle 208, a clinician may attach device 104 to the tether head assembly. The clinician may then load the device 104 into receptacle 208 via distal opening 212, and advance delivery catheter 204, with tether assembly 214 and device 104 therein, through introducer 202 and into the vasculature.

The tether head assembly may be loaded into receptacle 208 in an unlocked orientation relative to delivery catheter 204. In the unlocked orientation, locking element(s) on the tether head assembly may not interface with locking element(s) on a locking collar within delivery catheter 204, e.g., such that the tether head assembly may move unimpeded within the catheter lumen of delivery catheter 204 and along longitudinal axis 201 of delivery catheter 204. Movement of the tether head assembly along longitudinal axis 201 may cause device 104 to protrude from distal opening 212 of receptacle 208 or retract into receptacle 208. Once the clinician loads the tether head assembly into receptacle 208, the clinician may rotate the tether head assembly about longitudinal axis 201 to rotate tether head assembly from the unlocked orientation to the locked orientation. When the tether head assembly is in the locked orientation, the locking element(s) of the tether head assembly may interface with locking element(s) on the locking collar of delivery catheter 204 to inhibit distal movement of the tether head assembly and device 104 along longitudinal axis 201, e.g., movement of device 104 out of distal opening 212 of receptacle 208. When the tether head assembly is in the locked orientation, the tether head assembly and device 104 may freely move proximally along longitudinal axis 201, e.g., to facilitate flexure of delivery catheter 204 as the clinician navigates delivery catheter 204 within vasculature of the patient.

A proximal portion of tether assembly 214 (e.g., elongated member 218, a tether handle assembly 216) may be removably coupled to delivery catheter 204 (e.g., to handle 206, to a proximal portion of elongated body 210) via a locking feature, e.g., to prevent unintended rotation of tether head assembly and device 104 about longitudinal axis 201. The locking feature may include an elongated member disposed within catheter lumen of elongated body 210 and configured to be placed next to elongated member 218 inside catheter lumen. The locking feature may be permanently affixed to an inner surface. The locking feature and elongated member 218 may be configured to receive a locking component (e.g., a key, a locking pin). When the locking component is inserted through the locking feature and elongated member 218, the locking component may prevent rotation of elongated member 218 relative to the locking fixture.

Once the tether head assembly is in the locked orientation, the clinician may advance delivery catheter 204 into the vasculature to a target implant site in the vasculature, e.g., region 106 in heart 102. The clinician may rotate the tether head assembly about longitudinal axis 201 from the locked orientation to the unlocked orientation, advance the tether head assembly and device 104 from distal opening 212 of receptacle 208, and implant device 104 into tissue at the target implant site. In some examples, the clinician implants device 104 via rotation of tether head assembly and device 104 about longitudinal axis 201 to cause first electrode 112 to puncture and advance into the tissue. The clinician may rotate the tether head assembly to the unlocked orientation as a part of a rotation to implant device 104. For example, the clinician rotates the tether head assembly and device 104 about longitudinal axis 201 in a same direction to unlock tether head assembly and to implant device 104 into the tissue. In other examples, the clinician rotates the tether head assembly about longitudinal axis 201 in a first direction to unlock tether head assembly from the locking collar and rotates the tether head assembly in a second, different direction to implant device 104 into the tissue.

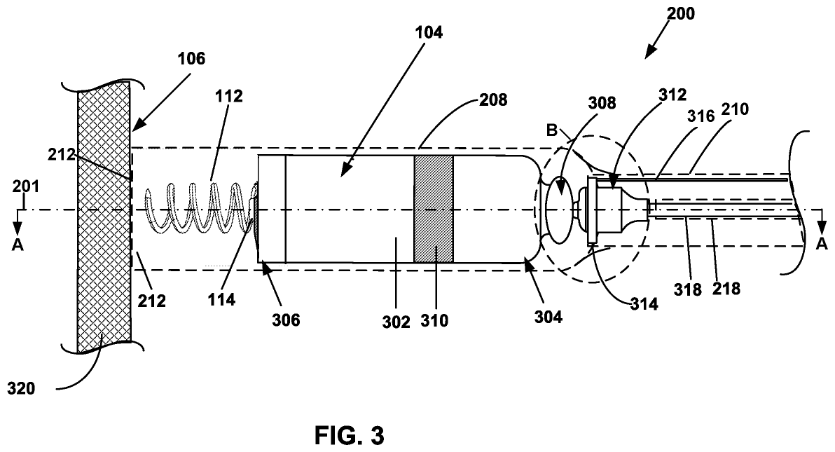

FIG. 3 is a conceptual drawing illustrating, in conjunction with tissue 320 of heart 102, a distal portion of medical device delivery system 200 carrying an example device 104. Device 104 may be a pacemaker device having a housing 302 that contains electronic components suitable for performing a variety of pacing functions. However, medical devices configured to deliver other types of electrical therapy to the patient or to perform other functions may be adapted for use with delivery system 200. Device 104 may include an attachment member 308 at a proximal end 304 thereof and fixation members (e.g., first electrode 112) at a distal end 306 thereof. Tether head assembly 312 of tether assembly 214 may be configured to receive and retain attachment member 308, as further discussed below.

In some examples, device 104 includes housing 302 defining a proximal end 304 and a distal end 306. Housing 302 may contain a pulse generator and an associated power supply (not shown) and electrodes 112, 114 which may be positioned at distal end 306 of housing 302 and which may be electrically coupled to the pulse generator of device 104 via a hermetically sealed feedthrough assembly (not shown). Housing 302 may be formed from any suitable biocompatible and biostable metal. For example, housing 302 may be formed from titanium and may be overlaid with an insulative layer (e.g., a medical grade polyurethane, parylene, or silicone). In some examples, device 104 may include a housing electrode 310, which may be formed by removing a portion of the insulative layer to expose a metallic surface defined by housing 302. In such examples, housing electrode 310 of device 104 may function in conjunction with electrodes 112, 114, such as for bipolar pacing and sensing.

FIG. 3 illustrates distal opening 212 of receptacle 208 of delivery catheter 204 pressed against tissue 320 at target implant region 106 of heart 102. When a clinician is satisfied with the positioning of receptacle 208 with respect to tissue 320, e.g., that longitudinal axis 201 of receptacle 208 is generally orthogonal to a plane defined by tissue 320, and that receptacle 208 pressed sufficiently against/into tissue 320 such that first electrode 112 of device 104 will deploy into tissue 320, the clinician may advance device 104 towards distal opening 212 using tether assembly 214, e.g., by using tether handle assembly 216 to advance tether assembly 214 distally relative to delivery catheter 204.

First electrode 112 may be configured to embed into tissue 320, and in some cases pull device 104 through distal opening 212 of receptacle 208, when advanced through distal opening 212.

Device 104 may, for a time, remain attached to tether assembly 214 by attachment member 308 and tether head assembly 312 while fixed to tissue 320 by first electrode 112. Thus, the clinician may be able to test the fixation of device 104 at the implant site and/or device 104 from the implant site and back into receptacle 208 for repositioning at a more suitable site, if necessary. Once satisfied with the implantation of device 104, the clinician can separate tether head assembly 312 from attachment member 308 and move tether assembly 214 proximally, as described in greater detail below, and then withdraw delivery catheter 204 and tether assembly 214 from the patient through introducer 202.

Tether assembly 214 may include an elongated member 218 coupled to a proximal end of tether head assembly 312. Elongated member 218 may retain a pull wire 318 disposed within a lumen of elongated member 218. Pull wire 318 may be attached at a distal end to tether head assembly 312 and attached at a proximal end thereof to tether handle assembly 216. The clinician may apply force to an actuator of tether handle assembly 216 to cause tether head assembly 312 to move from a closed position, in which attachment member 308 is retained within tether head assembly 312, to an open position in which attachment member 308 may be released from tether head assembly 312. With tether head assembly 312 in the open position, the clinician may proximally move tether assembly 214 to remove attachment member 308 from tether head assembly 312, leaving device 104 fixed at target implant region 106.

A clinician may secure attachment member 308 of device 104 to tether head assembly 312 by pressing attachment member 308 into a passageway defined by tether head assembly 312, thereby opening tether head assembly 312 from a first (e.g., closed) position to a second (e.g., open) position and advancing attachment member 308 through the passageway until attachment member 308 is received within tether head assembly 312.

A clinician may secure attachment member 308 of device 104 to tether head assembly 312 at the time of a medical procedure to deliver device 104. In addition, the clinician may release device 104 from tether head assembly 312 without cutting a portion of tether assembly 214. In some examples, tether head assembly 312 thus may reduce or eliminate drawbacks that may be associated with other types of tether mechanisms, such as tension associated with pulling on such other tether mechanisms (e.g., a loop of string or similar material), potential twisting or binding of such other tether mechanisms, or the like. The re-usability of tether assembly 214 may mitigate shelf life considerations with respect to tether assembly 214, delivery system 200, and device 104, such as in examples in which device 104 includes a drug eluting component with a finite shelf life. For example, tether assembly 214 and/or delivery system 200 may not necessarily be associated with a finite shelf life when packaged separately from device 104.

Once the clinician secures attachment member 308 of device 104 to tether head assembly 312, the clinician may retract device 104 and tether head assembly 312 proximally within receptacle 208. The clinician may align locking element(s) on tether head assembly 312 with locking element(s) on locking collar 314 of delivery catheter 204. Locking collar 314 may be disposed at a proximal end of or proximal to receptacle 208. In some examples, locking collar 314 may be disposed at a distal end of elongated body 210, e.g., at a location along delivery catheter 204 distal to elongated body 210 and proximal to receptacle 208. Locking collar 314 may be coupled to elongated member 316. Elongated member 316 may be configured to transmit a force from handle 206 to locking collar 314, e.g., to cause locking collar 314 and receptacle 208 to deflect relative to longitudinal axis 301. The clinician may deflect locking collar 314 and receptacle 208 from longitudinal axis 201 to facilitate navigation of a distal portion of delivery catheter 204 around curvatures in the vasculature of the patient.

Locking collar 314 may include locking element(s) that may interface with corresponding locking element(s) on tether head assembly 312 to prevent unintended distal movement of tether head assembly 312 relative to delivery catheter 204. By inhibiting unintentional distal movement of tether head assembly 312 via the interface between locking elements on tether head assembly 312 and on locking collar 314, the clinician may prevent unintended effects resulting from protrusion of distal end 306 of medical device 104, e.g., some or all of first electrode 112, from distal opening 212 of receptacle 208.

Locking element(s) on locking collar 314 may be configured to interface with locking element(s) on tether head assembly 312 only when tether head assembly 312 is in a specific orientation (e.g., in a locked orientation) relative to locking collar 314. The clinician may transition tether head assembly 312 between an unlocked orientation and a locked orientation by rotating tether head assembly 312 about longitudinal axis 201 via elongated member 218. Tether assembly 214 (e.g., a proximal portion of elongated member 218 and/or tether handle assembly 216) may include a first set or markers that may be aligned with corresponding markers on delivery catheter 204 (e.g., on a proximal portion of elongated body 210 and/or on handle 206) to indicate the orientation of tether head assembly 312 relative to locking collar 314. In some examples, the clinician determines that tether head assembly 312 is in a first orientation based on a determination that the first set of markers aligns with a second set of markers on delivery catheter 204 and determine that tether head assembly 312 is in an unlocked orientation based on a determination that the first set of markers aligns with a third set of markers on delivery catheter 204.

In some examples, locking element(s) on locking collar 314 includes one or more locking protrusions and locking element(s) on tether head assembly 312 includes one or more locking recesses, each locking recess being configured to receive a locking protrusion. In some examples, locking element(s) on locking collar 314 incudes one or more locking recesses and locking element(s) on tether head assembly 312 include one or more locking protrusions. In some examples, locking element(s) on locking collar 314 and tether head assembly 312 may be other components configured to interface with each other to inhibit distal movement of tether head assembly 312.

During delivery of device 104 to region 106 via delivery system 100, the clinician may advance receptacle 208 into contact with tissue 320 of heart 102 prior to engaging first electrode 112 with tissue 320 of heart 102. In such examples, tether head assembly 312 is in the locked orientation prior to first electrode 112 engaging tissue 320. The clinician then may determine whether receptacle 208 and device 104 are properly positioned at region 106 prior to engaging first electrode 112 with tissue 320 of heart 102. In some examples, the clinician may determine whether receptacle 208 and device 104 are properly positioned relative to heart 102 based on an impedance or other electrical signal sensed via an electrical path including device 104 (e.g., housing 302 or an electrode 310), attachment member 308, and one or more components of tether assembly 214 (e.g., one or more components of tether head assembly 312). In addition to device 104, another electrode of the electrical path may be a reference electrode attached to the patient, or inside the patient but located outside of receptacle 208. In some examples, relatively higher impedance may be indicative of receptacle 208 being positioned flush against, and with adequate depth in, tissue 320 of heart 102, which may be desirable for proper fixation.

The clinician may rotate tether head assembly 312 about longitudinal axis 201 from the locked orientation to the unlocked orientation to enable distal movement of device 104 and or tether head assembly 312. The clinician may then deploy device 104 by rotating tether head assembly 312 and device 104 about longitudinal axis 201 to cause first electrode 112 to pierce and engage with tissue 320 at region 106. In some examples, where the example medical device includes other fixation components (e.g., fixation tines), the clinician may advance the device out of distal opening 212 of receptacle 208 to cause the fixation components to engage with tissue 320. After deployment of device 104 from receptacle 208, with device 104 fixed to tissue 320, an impedance or electrical signal may also indicate the quality of the fixation of device 104 to tissue 320, e.g., based on variations of the impedance during a “tug test” in which a clinician pulls on tether assembly 214 while attached to device 104 and while device 104 is fixed to tissue 320. Some examples may employ any of the techniques for testing the spatial relationship of receptacle 208 and/or device 104 to tissue 320, and for testing fixation of device 104 to tissue 320.

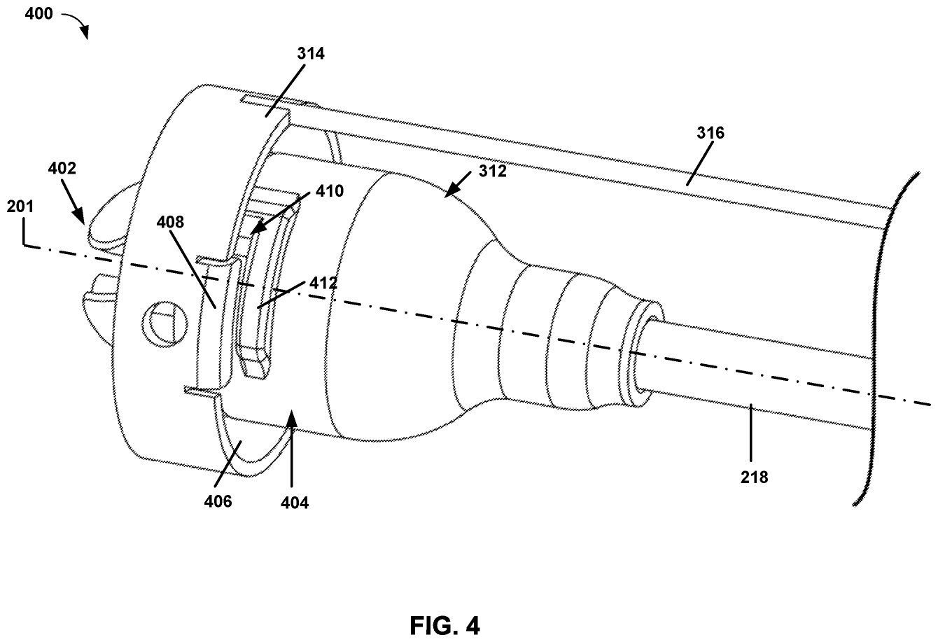

FIG. 4 is a perspective diagram illustrating an example tether head assembly 312 and locking collar 314 of the example medical device delivery system 200 of FIG. 2 in a locked orientation 400. Tether head assembly 312 may include a locking element 410 disposed on the outer surface of tether head assembly 312. Locking collar 314 may include one or more locking elements 408 disposed on locking collar 314. Each locking element 408 may be configured to interface with a corresponding locking element 410 on tether head assembly 312 to inhibit distal movement of tether head assembly 312 along longitudinal axis 201.

Locking collar 314 may be coupled to elongated member 316 and may be configured to receive a force from handle 206 of delivery catheter 204 via elongated member 316. Locking collar 314 may include an annular body 406 defining an inner surface defining an inner lumen of locking collar 314. The inner lumen of locking collar 314 may be size to facilitate unimpeded movement of tether head assembly 312 through the inner lumen. The inner lumen of locking collar 314 may define an inner diameter of about 3.43 millimeters (mm) (e.g., about 0.135 inches (in)). Locking collar 314 may include one or more locking elements 408 disposed around a perimeter of locking collar 314. In examples where locking collar 314 includes two or more locking elements 408, the locking elements 408 may be equally distributed around the perimeter of locking collar 314. In some examples, two or more locking elements 408 may be biased around a portion of locking collar 314, e.g., two or more locking elements 408 may be in close proximity around elongated member 316, and/or around a portion of locking collar 314 opposite elongated member 316 than other locking elements 408.

Each locking element 408 may be formed from a same or different material than locking collar 314. The material may include, but is not limited to, stainless steel. In some examples, a manufacturer forms portions of annular body 406 into tabs and folds the tabs radially inwards towards longitudinal axis 201 to form locking elements 408. In some examples, the manufacturer may remove or add material to annular body 406 to form locking elements 408. Locking elements 408 may include locking protrusions, as illustrated in FIG. 4, locking recesses, locking wedges, locking grooves, or other components configured to interface with locking element 410 on tether head assembly 312 to prevent distal movement of tether head assembly 312 along longitudinal axis 201.

Tether head assembly 312 may include retainers 402 configured to be removably coupled to attachment member 308 on device 104. Retainers 402 may include an inner retainer and outer retainer that may be operated by a clinician to retain or release attachment member 308. Retainers 402 are disposed within a sheath 404 defining an outer surface of tether head assembly 312. One or more locking elements 410 are disposed on the outer surface of tether head assembly 312. Tether head assembly 312 may include one or more locking elements 410 on the outer surface of tether head assembly Each locking element 410 is configured to interface with a corresponding locking element 408. Each locking element 410 may be identical to another locking element 410 or may be different from another locking element 410. Locking element 410 may include a locking recess, as illustrated in FIG. 4, a locking protrusion, or other locking components. In some examples, as illustrated in FIG. 4, the manufacturer may add material to the outer surface of tether head assembly 312 (e.g., via an additive manufacturing technique, via a metal injection technique) to form a protrusion 412 defining locking element 410. Protrusion 412 may define a rectangular shape, an “L” shape, a “U” shape, a horseshoe shape, two parallel rectangular shapes separated by a distance greater than or equal to a width of locking element 408 along longitudinal axis 201, or other shape(s).

The sidewalls of locking element 410 (e.g., the sidewalls of protrusion 412 defining locking element 410) may interface with sidewalls of locking element 408 to prevent distal movement of tether head assembly 312 along longitudinal axis 201. Locking elements 408, 410 may allow for proximal movement of tether head assembly 312 along longitudinal axis 201 and the return of tether head assembly 312 to a distalmost location allowed by locking elements 408, 410, e.g., to facilitate flexure of delivery catheter 204 within the vasculature of the patient. Locking elements 408, 410 may define heights and/or depths such that each pair of locking elements 408, 410 radially and circumferentially overlap when tether head assembly 312 is in locked orientation 400.

In some examples, one or more of locking elements 408, 410 may be configured to elastically deform and allow distal movement of tether head assembly 312 while tether head assembly 312 is in locked orientation 400. Locking elements 408, 410 may elastically deform in response to the clinician applying a threshold amount of force to tether head assembly 312 (e.g., via elongated member 218) along longitudinal axis 201.

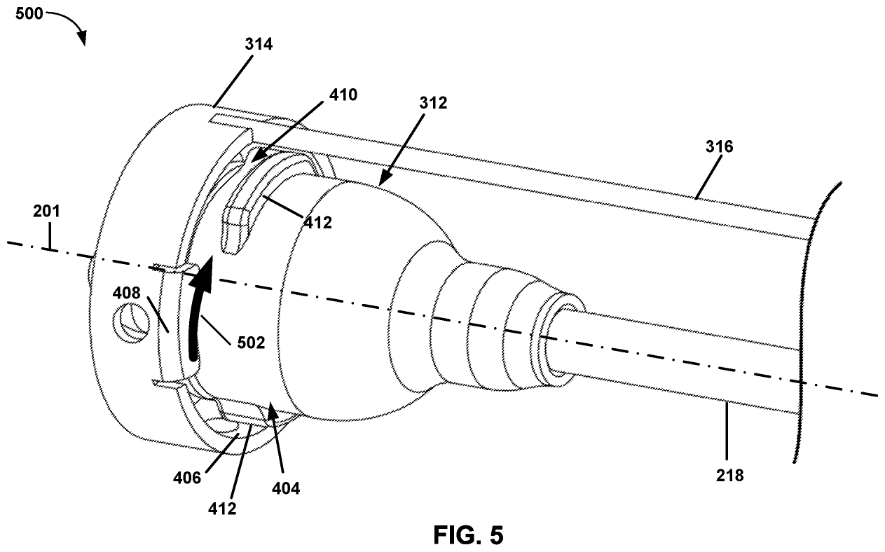

FIG. 5 is a perspective diagram illustrating the example tether head assembly 312 and locking collar 314 of FIG. 4 in an unlocked orientation 500. In unlocked orientation 500, locking elements 408 on locking collar 314 do not interface with locking elements 410 on tether head assembly 312. The clinician may freely advance tether head assembly 312 distally along longitudinal axis 201 and out of distal opening 212 of receptacle 208. In unlocked orientation 500, locking elements 408 do not circumferentially overlap locking elements 410, e.g., such that when locking elements 408 and locking elements 410 are at a same longitudinal position relative to longitudinal axis 201, locking elements 408 do not come into contact with locking elements 410.

The clinician may rotate tether head assembly 312 from locked orientation 400 to unlocked orientation 500 by rotating tether head assembly 312 about longitudinal axis 201 in a direction 502. Direction 502 may be in a clockwise or a counter-clockwise direction. The clinician may determine a degree of rotation of tether head assembly 312 based on markings on tether assembly 214 and on delivery catheter 204. For example, a first set of markings on tether assembly 214 aligns with a second set of markings on delivery catheter 204 when tether head assembly 312 is in locked orientation 400 and the first set of markings align with a third set of markings on delivery catheter 204 when tether head assembly 312 is in an unlocked orientation 500.

The clinician may implant device 104 by rotating device 104 about longitudinal axis 201 in direction 502. In such examples, the clinician rotates tether head assembly 312 to unlocked orientation 500 as a part of implanting device 104. First electrode 112 of device 104 may include an elongated body defining a helix wound around longitudinal axis 201 in direction 502, e.g., to facilitate incorporating the rotation of tether head assembly 312 to unlocked orientation 500 and implantation of device 104 into a single act of rotating tether assembly 214. In some examples, the clinician may implant device by rotating device 104 about longitudinal axis 201 in a direction opposite to direction 502. The clinician may rotate device 104 and tether head assembly 312 about longitudinal axis 201 in direction 502 to rotate tether head assembly 312 to unlocked orientation 500. The clinician may then advance device 104 and tether head assembly 312 distally along longitudinal axis 201 until locking elements 410 on tether head assembly 312 are distal to locking elements 408 on locking collar 314. The clinician may then rotate device 104 and tether head assembly 312 about longitudinal axis 201 in an direction opposite to direction 502 to implant device 104 in tissue 320 of the patient.

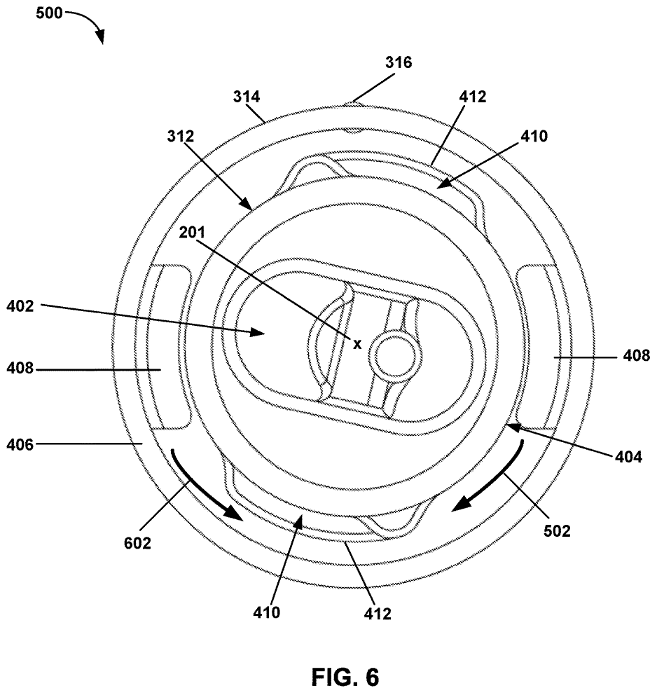

FIG. 6 is a perspective diagram illustrating a front view of the example tether head assembly 312 and locking collar 314 of FIG. 4 in unlocked orientation 500. FIG. 6 illustrates a front view from a reference plane orthogonal to longitudinal axis 201 and distal to tether head assembly 312 and locking collar 314. As illustrated in FIG. 6, locking collar 314 may include two locking elements 408 and tether head assembly 312 may include two locking elements 410. In other examples, locking collar 314 may include one or three or more locking elements 408 and tether head assembly 312 may include one or three or more locking elements 410.

Locking elements 408 may be identical and locking elements 410 may be identical, e.g., such that any locking element 410 may interface with any locking element 408 when tether head assembly 312 is in locked orientation 400. In some examples, one or more locking elements 408 may be different from another locking element 408 and one or more locking elements 410 may be different from another locking element 410, e.g., such that each locking element 408 may only interface with a specific locking element 410, and vice versa. In such examples, tether head assembly 312 may only be in locked orientation 400 when tether head assembly 312 is positioned at a specific orientation relative to locking collar 314. Different locking elements 408 and different locking elements 410 may define different dimensions and/or shapes, e.g., such that each locking element 408 may only interface with a corresponding locking element 410, and vice versa.

As illustrated in FIG. 6, locking elements 408 be equally distributed around a perimeter of locking collar 314. In other examples, locking elements 408 may be biased towards a specific portion of locking collar 314, e.g., around elongated member 316, around a portion of locking collar 314 opposite elongated member 316. Locking elements 410 may be distributed around an outer diameter of tether head assembly 312 such that when tether head assembly 312 is in locked orientation 400, one or more locking elements 408 may at least partially circumferentially overlap one or more locking elements 410 and inhibit distal movement of tether head assembly 312.

The clinician may rotate tether head assembly 312 about longitudinal axis 201 in direction 502 to rotate tether head assembly 312 to unlocked orientation 500. The clinician may rotate tether head assembly 312 about longitudinal axis 201 in direction 602 to rotate tether head assembly 312 to locked orientation 400. Direction 602 may be opposite to direction 502. As illustrated in FIGS. 4-6, protrusions 412 may define a single opening into each locking recess defining a locking element 410. In such examples, locking element 410 may inhibit further rotation of tether head assembly 312 in direction 602 when tether head assembly 312 is in locked orientation 400. In other examples, locking recesses defining locking element 408 or locking element 410 may include locking recesses with openings at both ends of each locking recess. In such examples, the clinician may rotate tether head assembly 312 between locked orientation 400 and unlocked orientation 500 by rotating tether head assembly 312 in either direction 502 or direction 602.

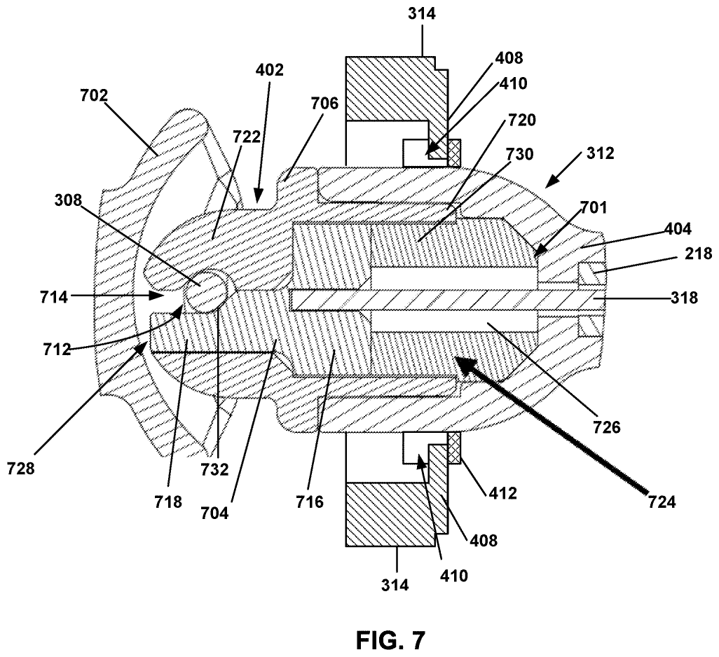

FIG. 7 is a cross-sectional view of the highlighted portion B of FIG. 3 including the tether head assembly 312, locking collar 314, and a proximal portion of device 104, where the cross-section is taken along line A-A of FIG. 3 in a plane parallel to longitudinal axis 201 of the tether assembly 214 and longitudinal axis 201 of device 104. As illustrated in FIG. 7, retainers 402 of tether head assembly 312 includes inner retainer 704 and outer retainer 706. Retainers 704, 706 may removably secure attachment member 308 to tether head assembly 312. FIG. 7 illustrates one example embodiment of tether head assembly 312. Other example tether head assembly 312 are described in U.S. Pat. No. 11,331,475, filed on Apr. 13, 2020 and granted on May 17, 2022 and entitled “Tether Assemblies for Medical Device Delivery Systems,” the entirety of which is herein incorporated by reference.

Sheath 404 of tether head assembly 312 is attached to the distal end of elongated member 218 of tether assembly 12. Pull wire 318 extends through a lumen defined by elongated member 218 and into cavity 701 defined by sheath 404. Elastically-compressible member 724, proximal portion 716 of inner retainer 704, and proximal portion 720 of outer retainer 706 are disposed within cavity 701, with a distal portion 730 of elastically-compressible member 724 and proximal portion 716 of inner retainer 704 received within a channel defined by proximal portion 720 of outer retainer 706. Pull wire 318 extends through lumen 726 defined by elastically-compressible member 724, and is connected to inner retainer 704, e.g., fixedly received within proximal portion 716 of inner retainer 704. Various components of system 200 and tether assembly 214 may be connected by any of a variety of techniques, such as welding, crimping, threading, reflowing, bonding, adhesives, or friction fits.

Distal portion 718 of inner retainer 704 extends into distal portion 722 of outer retainer 706 to contribute to define receptacle 712. When attachment member 308 is in receptacle 712, inner retainer 704 may be in a first position such that distal portion 718 of inner retainer 704 extends into passageway 714 to reduce the size of the passageway such that a thickness or depth of the passageway 714 is smaller than a thickness of attachment member 308 of device 104. In the first position of inner retainer 704, distal portion 718 of inner retainer 704 may be disposed within a groove defined by distal portion 722 of outer retainer 706. When inner retainer 36 is in the first position, elastically-compressible member 724 may be in a relaxed, or lower kinetic energy state.

Attachment member 308 of device 104 may be included as part of a structure that provides a variety of features supporting a variety of functions related to delivery and retrieval of device 104. Attachment member 308 may be formed within and joined to housing 302 of device 104 via a shroud structure 702. Attachment member 308 may include a pin or a strut welded to or otherwise fixedly attached to shroud structure 702. Attachment member 308 provides an elongate holding surface that is spaced apart from housing proximal end 304 of housing 302 and that extends along a length substantially orthogonal to a longitudinal axis 201 of device 104.

Shroud structure 702 may define a cavity with an opening and attachment member 308 may span and be exposed at the opening. Attachment member 308 may be welded at either end to opposing sides of shroud structure 702. Distal portion 722 of outer retainer 706 may be configured to enter or otherwise interact with shroud structure 702 when attachment member 308 is received within passageway 714 and receptacle 712. The configuration of shroud structure 702 and distal portion 722 of outer retainer 706 may selectively inhibit or allow relative motion of device 104 and tether assembly 214 in a variety of directions. It should be understood that shroud structure 702 and attachment member 308 are provided for example only, and that a variety of other attachment members may be configured to be attached to tether assemblies as described herein.

When inner retainer 704 is in a second position, attachment member 308 may enter receptacle 712 via passageway 714. Inner retainer 704 may be moved to the second position by a proximally directed force. The proximally directed force may be provided by a pulling force from pull wire 318 or a pushing force on distal end 728 of inner retainer 704 as attachment member 308 is pushed through passageway 714 and into receptacle 712. Movement of inner retainer 704 to the second position may compress elastically-compressible member 724, e.g., such that distal portion 730 is no longer located within the channel defined by proximal portion 720 of outer retainer 706.

When in this compressed state, elastically-compressible member 724 may retain elastic energy to be released by expanding in the direction of its longitudinal axis (e.g., longitudinal axis 201) to the expanded or relaxed state as illustrated in FIG. 7, thereby moving inner retainer 704 from the second position to the first position illustrated in FIG. 7. FIG. 7 illustrates attachment member 308 held within receptacle 712 defined by outer retainer 706 and inner retainer 704 in the first position. Receptacle 712 is configured, e.g., sized and shaped, to retain attachment member 308 while allowing distal portion 718 of inner retainer 704 to move past attachment member 308, e.g., through passageway 714. As illustrated in FIG. 7, at least third portion 732 of distal portion 718 of inner retainer 704 may contact attachment member 308 of device 104 when attachment member 308 is positioned within receptacle 712, e.g., when inner retainer 704 is in the first position. As described herein, third portion 732 may secure attachment member 308 within receptacle 712 and help ensure substantially constant physical contact between attachment member 308 and at least third portion 732 of inner retainer 704. The physical contact between attachment member 308 and inner retainer 704 enabled by third portion 732 may provide substantially constant electrical contact for conduction of electrical signals, e.g., for impedance monitoring, from device 104 to a proximal portion of tether assembly 214.

As illustrated in FIG. 7, an inner lumen of locking collar 314 is configured to receive tether head assembly 312. Locking elements 408 of locking collar 314 may interact with locking elements 410 to prevent distal movement of tether head assembly 312 relative to locking collar 314. Locking elements 408, 410 radially overlap such that sidewalls of locking elements 408 contacts the sidewalls of corresponding locking elements 410 to prevent distal movement of therapy head assembly 312. While FIG. 7 illustrated locking elements 408 as defining locking protrusions and locking elements 410 as including protrusions defining locking recess, other example systems 200 may include locking elements 408 defining locking recesses and locking elements 410 defining locking protrusions.

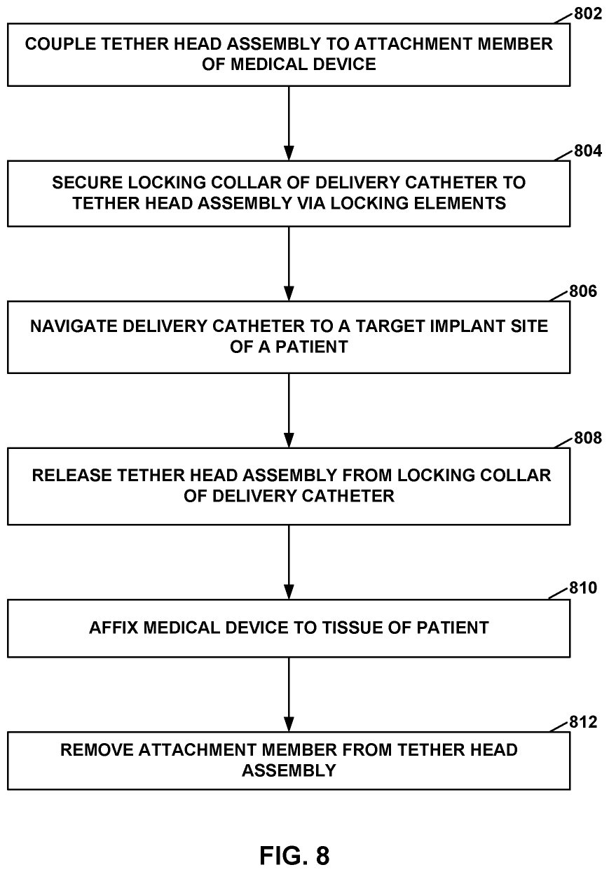

FIG. 8 is a flowchart illustrating an example process of implanting a medical device 104 with the medical device delivery system 200 of FIG. 2. While the example process of FIG. 8 is described primarily with reference to system 200 and components of system 200 as illustrated in FIGS. 1-7, the example techniques of FIG. 8 may be applied with any other example systems described herein.

A clinician may couple tether head assembly 312 to attachment member 308 of medical device 104 (802). The clinician may insert tether head assembly 312 and elongated member 218 into a catheter lumen of delivery catheter 204 of system 200. Catheter lumen may extend along longitudinal axis 201 through handle 206 and elongated body 210 to a proximal end of receptacle 208. The clinician may insert tether head assembly 312 through the catheter lumen until tether head assembly 312 protrudes through distal opening 212 of receptacle 208. The clinician may maintain tether head assembly 312 in an unlocked orientation 500 as the clinician advances tether head assembly 312 through the catheter lumen of delivery catheter 204 to prevent locking collar 314 from impeding movement of tether head assembly 312, e.g., via contact between locking elements 408 on locking collar 314 and locking elements 410 on tether head assembly 312.

Once tether head assembly 312 protrudes from distal opening 212, the clinician may insert attachment member 308 of medical device 104 into tether head assembly 312. The clinician may cause inner retainer 704 to retract from a first position to a second position to expand a passageway 714 leading to a receptacle 712 of tether head assembly 312. Receptacle 712 may be defined by inner retainer 704 and outer retainer 706 of tether head assembly 312. The clinician may retract inner retainer 704 to the second position by retracting a pull wire 318 disposed within elongated member 218. In some examples, the clinician may press a distal end of tether head assembly 312 (e.g., a distal end 728 of inner retainer 704) against attachment member 308 and/or shroud structure 702 of device 104 to cause inner retainer 704 to retract to the second position.

Retraction of inner retainer 704 to the second position causes elastically-compressible member 724 may compress and store elastic energy. Once attachment member 308 completely enters receptacle 712 of tether head assembly 312, elastically-compressible member 724 may release the stored elastic energy and advance inner retainer 704 from the second position to the first position. When inner retainer 704 is in the first position, inner retainer 704 restricts passageway 714, e.g., to prevent attachment member 308 from exiting receptacle 712 via passageway 714. Attachment member 308 may be securely retained within receptacle 712 via contact between attachment member 308 and distal portion 722 of outer retainer 706 and third portion 732 of inner retainer 704.

The clinician may secure locking collar 314 of delivery catheter 204 to tether head assembly 312 via locking elements 408, 410 (804). The clinician may secure locking collar 314 to tether head assembly 312 to prevent unintentional distal movement of tether head assembly 312 and device 104, e.g., due to a force of an injection of a fluid into vasculature of the patient via the catheter lumen of delivery catheter 204, due to a reduction in overall length of delivery catheter 204 due to deflection of delivery catheter 24 within vasculature of the patient. The clinician may retract tether head assembly 312 and device 104 into receptacle 208 until locking elements 410 align with (e.g., are at a same longitudinally position along longitudinal axis 201) locking elements 408 on locking collar 314. Locking collar 314 may be disposed at a proximal portion of receptacle 208 or may be disposed within elongated body 210 at a position proximal to receptacle 208. Locking collar 314 may define an inner lumen in fluid communication with an inner volume of receptacle 208 and with the catheter lumen. The inner lumen may be sized to receive tether head assembly 312.

Once locking elements 408 and locking elements 410 are aligned, the clinician may rotate tether head assembly 312 about longitudinal axis 201 from unlocked orientation 500 to locked orientation 400. When tether head assembly 312 is in locked orientation 400, locking elements 408 interface with locking elements 410 to inhibit distal movement of tether head assembly 312 and device 104 out of receptacle 208. Locking elements 408, 410 may include, but are not limited to, locking protrusions (e.g., extending along a plane orthogonal to longitudinal axis 201), locking recesses, locking wedges, locking grooves, or the like. In some examples, locking element 408 includes locking protrusion extending from annular body 406 of locking collar 314 radially inwards towards longitudinal axis 201 and locking element 410 includes locking recesses disposed on an outer surface of tether head assembly 312. Tether head assembly 312 may include protrusions extending from the outer surface of tether head assembly 312 and form the locking recesses. In some examples, locking element 408 includes locking protrusions disposed on an inner surface of annular body 406 of locking collar 314 and locking element 410 includes locking protrusions extending from an outer surface of tether head assembly 312 and away from longitudinal axis 201.

When tether head assembly 312 is in locked orientation 400, locking elements 408 and the corresponding locking elements 410 may circumferentially and radially overlap such that locking elements 408 (e.g., sidewalls of locking protrusions) contact the corresponding locking elements 410, sidewalls of locking recesses, sidewalls of protrusions defining locking recesses to inhibit distal movement of tether head assembly 312. In some examples, locking elements 408 may be identical and locking elements 410 may be identical such that any locking element 408 may interface with any locking element 410. In some examples, one or more locking elements 408 may be different from another locking element 408 and one or more locking elements 410 may be different from another locking element 410 such that when tether head assembly 312 is in locked orientation 400, tether head assembly is in a specific orientation relative to delivery catheter 204. The specific orientation may allow the clinician to precisely place device 104 and/or a piercing tip of device 104 against tissue of the patient.

The clinician may secure elongated member 218 to delivery catheter 204, e.g., to a locking feature on handle 206 and/or on a proximal portion of elongated body 210. The locking feature may be disposed within the catheter lumen of delivery catheter 204 next to elongated member 218. The clinician may insert a locking component through the locking feature and elongated member 218 to prevent rotation of elongated member 218 relative to delivery catheter 204. Securing elongated member 218 to delivery catheter 204 may inhibit unintended rotation of tether head assembly 312 within delivery catheter 204, thereby inhibiting unintentional transition of tether head assembly 312 between locked orientation 400 and unlocked orientation 500. In the locked orientation 400, tether head assembly 312 may retract proximally along longitudinal axis 201, e.g., to facilitate flexure and deflection of delivery catheter 204 as the clinician navigates delivery catheter 204 within vasculature of patient.

The clinician may navigate delivery catheter 204 to a target implant site of the patient (e.g., region 106 in heart 102) (806). The clinician may advance delivery catheter 204 into vasculature of the patient via introducer 202. The clinician may advance delivery catheter 204 through the vasculature of the patient to the target implant site and may inject fluid into the vasculature at various locations to aid in visualization of delivery catheter 204. Locking collar 314 may prevent any unintended distal movement of tether head assembly 312 and/or of device 104 within delivery catheter 204 as a result of forces acting on tether head assembly 312 by the fluid or as a result of flexure of delivery catheter 204. The clinician may place distal opening 212 of receptacle 208 in contact with tissue 320 at region 106. The clinician may determine proper placement of receptacle 208 at region 106 via sensed electrical signals (e.g., impedance).

The clinician may release tether head assembly 312 from locking collar 314 of delivery catheter 204 (808). The clinician may remove locking element from locking feature and elongated member 218 to allow rotation of elongated member 218 relative to delivery catheter 204. The clinician may rotate elongated member 218 to cause tether head assembly 312 to rotate in a direction 502 from locked orientation 400 to unlocked orientation 500. The clinician may rotate elongated member 218 in a same direction 502 or in a different direction 602 to rotate tether head assembly 312 from unlocked orientation 500 to locked orientation 400 The clinician may determine a degree of rotation based on markings on delivery catheter 204 and elongated member 218. The clinician may rotate elongated member 218 until markings on elongated member 218 align with markings on delivery catheter 204 corresponding to unlocked orientation 500.

The clinician may affix medical device 104 to tissue of the patient (810). The clinician may place a distal tip (e.g., a distal tip of first electrode 112) of device 104 in contact with tissue 320 of the patient. The clinician may then rotate elongated member 218 to cause the distal tip of device 104 to pierce and advance into tissue 320 of the patient. The clinician may rotate elongated member 218 until the clinician determines that device 104 is at a desired depth within tissue 320 (e.g., until second electrode 114 is placed in contact with tissue 320 without piercing tissue 320). In some examples, wherein device 104 includes one or more fixation tines, the clinician may advance elongated member 218 distally through delivery catheter 204 to cause device 104 to protrude from receptacle 208 and deploy fixation tine(s) into tissue 320.

The clinician may sense electrical signals from tissue 320 and/or deliver test stimulation signals to tissue 320 via device 104. The clinician may adjust, based on the sensed electrical signals and/or on feedback from the test stimulation signals, the implantation location of device 104 until the clinician determines that the sensed electrical signals and/or the feedback from the test stimulation signals satisfy one or more tests (e.g., a “tug test”) or threshold conditions(s).

In some examples, the clinician may rotate tether head assembly 312 to unlocked orientation 500 and affix device 104 to tissue 320 in a single act of rotating elongated member 218. In some examples, the clinician may rotate elongated member 218 in a first direction (e.g., direction 502) to rotate tether head assembly 312 to unlocked orientation 500, advance tether head assembly 312 distal to locking collar 314, and rotate tether head assembly 312 in a second direction (e.g., direction 602) to implant device 104 within tissue 320.

The clinician may remove attachment member 308 from tether head assembly 312 (812). The clinician may retract pull wire 318 to cause inner retainer 704 to retract from the first position to the second position. Retrain of inner retainer 704 to the second location may cause passageway 714 to expand, e.g., due to proximal movement of distal portion 718 or inner retainer 704. The clinician may retract tether head assembly 312 proximally by retracting elongated member 218. The clinician may maintain inner retainer 704 in the second position while retracting tether head assembly 312 to cause attachment member 308 to exit receptacle 712 via passageway 714.

The following examples are illustrative of the techniques described herein.

Example 1: a system comprising: a delivery catheter comprising: an elongated body extending from a proximal end to a distal end along a longitudinal axis, wherein the elongated body defines a catheter lumen extending from the proximal end to the distal end; a receptacle extending distally from the distal end of the elongated body along the longitudinal axis, the receptable defining an inner volume configured to retain an implantable medical device, wherein the inner volume is in fluid communication with the catheter lumen; and a locking collar disposed at the distal end of the elongated body, the locking collar comprising a first locking element; and a tether assembly configured to be disposed within the catheter lumen, wherein the tether assembly comprises: a tether head assembly configured to receive an attachment member of the implantable medical device; and a second locking element disposed on an outer surface of the tether head assembly, wherein the second locking element is configured to interface with the first locking element of the locking collar to inhibit unintended distal movement of the implantable medical device within the receptacle.

Example 2: the system of example 1, wherein the tether assembly is configured to transition between a locked orientation and an unlocked orientation relative to the locking collar via rotation of the tether assembly about the longitudinal axis, wherein the locking collar is configured to inhibit distal movement of the tether head assembly when the tether assembly is in the locked orientation, and wherein the locking collar is configured to permit distal movement of the tether head assembly when the tether assembly is in the unlocked orientation.

Example 3: the system of example 2, wherein when the tether assembly is in the locked orientation relative to the locking collar, the first locking element of the locking collar interfaces with the second locking element of the tether assembly.

Example 4: the system of any of examples 2 and 3, wherein when the tether assembly is in the unlocked orientation relative to the locking collar, the first locking element of the locking collar does not interface with the second locking element of the tether assembly.

Example 5: the system of any of examples 2-4, wherein the first locking element comprises a locking protrusion extending radially inwards from the locking collar and towards the longitudinal axis, wherein the second locking element comprises a locking recess on the outer surface of the tether head assembly, and wherein when the tether assembly is in the locked orientation relative to the locking collar, the locking protrusion is disposed within the locking recess and interfaces with a sidewall of the locking recess to inhibit unintended distal movement of the implantable medical device within the receptacle.

Example 6: the system of example 5, wherein the retainer comprises a protrusion extending radially outwards away from the outer surface of the tether head assembly, and wherein the protrusion defines the locking recess.

Example 7: the system of any of examples 2-4, wherein the first locking element comprises a locking recess disposed on an inner surface of the locking collar, wherein the second locking element comprises a locking protrusion extending radially outwards away from the longitudinal axis and the outer surface of the tether head assembly, and wherein when the tether assembly is in the locked orientation relative to the locking collar, the locking protrusion is disposed within the locking recess and interfaces with a sidewall of the locking recess to inhibit unintended distal movement of the implantable medical device within the receptacle.

Example 8: the system of any of examples 2-7, wherein the tether assembly is configured to affix the implantable medical device within tissue of a patient via the rotation of the tether assembly about the longitudinal axis, and wherein the tether assembly is configured to transition from the locked orientation to the unlocked orientation as a part of affixing the implantable medical device within the tissue.

Example 9: the system of example 8, wherein the implantable medical device defines an elongated body defining a helix extending from a distal end of the implantable medical device, wherein the helix is wound in a first direction, and wherein the tether assembly is configured to rotate about the longitudinal axis in the first direction to transition from the locked orientation to the unlocked orientation.

Example 10: the system of any of examples 2-9, wherein the rotation of the tether assembly within the delivery catheter comprises a first rotation of the tether assembly, wherein the tether assembly is configured to affix the implantable medical device within tissue of a patient via a second rotation of the tether assembly about the longitudinal axis, wherein the first rotation is separate from the second rotation.

Example 11: the system of example 10, wherein the first rotation is in a first direction, and wherein the second rotation is in a second direction, the first direction being different from the second direction.

Example 12: the system of any of examples 1-11, wherein the tether assembly comprises an elongated member coupled to a proximal end of the tether head assembly, wherein the elongated member is disposed within the catheter lumen, and wherein the elongated member is configured to be affixed to a proximal portion of the elongated body of the delivery catheter via a locking feature disposed along the proximal portion of the elongated body.

Example 13: the system of example 12, wherein the locking feature is configured to inhibit unintended rotation of the tether assembly about the longitudinal axis.

Example 14: the system of any of examples 1-13, wherein the tether assembly further comprises a tether handle assembly coupled to the tether head assembly via an elongated member disposed within the catheter lumen, wherein the tether handle assembly is configured to cause movement of the tether head assembly along the longitudinal axis or rotation of the tether head assembly about the longitudinal axis.

Example 15: a tether head assembly comprising: an inner retainer coupled and extending distally from a distal end of an elongated member; an outer retainer defining a channel configured to receive the inner retainer, wherein the inner retainer and the outer retainer define a receptacle configured to receive an attachment member of a medical device; a sheath configured to receive the inner retainer and the outer retainer; and a first locking element disposed on an outer surface of the sheath, wherein the tether head assembly is configured to be disposed within a catheter lumen of an elongated body of a delivery catheter, wherein the elongated member is configured to transmit a force from a proximal end of the elongated member to the inner retainer to move the tether head assembly within the catheter lumen of the delivery catheter. and wherein the first locking element is configured to interface with a second locking element disposed within an inner lumen to inhibit unintended movement of the tether head assembly towards a distal end of the elongated body of the delivery catheter.

Example 16: the tether head assembly of claim 15, wherein the force comprises a rotational force, and wherein the tether head assembly is configured to rotate about a longitudinal axis of the tether head assembly in response to the rotational force, wherein the tether head assembly is configured to transition between a locked orientation and an unlocked orientation relative to the delivery catheter as a result of the rotation of the tether head assembly about the longitudinal axis, wherein the first locking element of the tether head assembly is configured to interface with the second locking element of the delivery catheter when the tether head assembly is in the locked orientation, and wherein the first locking element of the tether head assembly is configured to not interface with the second locking element of the delivery catheter when the tether head assembly is in the unlocked orientation.

Example 17: the tether head assembly of example 16, wherein the tether head assembly is configured to rotate about the longitudinal axis in a first direction about the longitudinal axis to affix the medical device within tissue of a patient, wherein the tether head assembly is configured to rotate about the longitudinal axis in a second direction about the longitudinal axis to transition from the locked orientation to the unlocked orientation.

Example 18: the tether head assembly of example 17, wherein the first direction and the second direction are in opposite directions.

Example 19: the tether head assembly of any of examples 16-18, wherein the first locking element comprises a locking recess on the outer surface of the sheath, wherein the second locking element comprises a locking protrusion extending from an inner surface of the elongated body defining the catheter lumen and towards the longitudinal axis, and wherein locking recess is configured to receive the locking protrusion when the tether assembly is in the locked orientation.

Example 20: the tether head assembly of example 19, wherein the sheath defines a projection extending radially away from the outer surface of the sheath, and wherein the protrusion defines the locking recess.

Example 21: the tether head assembly of any of examples 16-20, wherein the first locking element comprises a protrusion extending radially away from the outer surface of the sheath, wherein the second locking element comprises a locking recess dispose on an inner surface of the elongated body defining the catheter lumen, and wherein the protrusion is configured to be disposed within the locking recess when the tether assembly is in the locked orientation.

Example 22: a catheter comprising: an elongated body extending from a proximal end to a distal end along a longitudinal axis, wherein the elongated body defines a catheter lumen extending from the proximal end to the distal end; a receptacle extending distally from the distal end of the elongated body along the longitudinal axis, the receptable defining an inner volume configured to retain an implantable medical device, wherein the inner volume is in fluid communication with the catheter lumen; and a locking collar disposed at the distal end of the elongated body, the locking collar comprising a first locking element, wherein the first locking element is configured to interface with a second locking element on an outer surface of a tether head assembly disposed within the catheter lumen and attached to the implantable medical device to prevent unintended distal movement of the implantable medical device along the longitudinal axis.

Example 23: the catheter of example 22, where the locking collar is configured to transition between a locked configuration and an unlocked configuration in response to rotation of the tether head assembly about the longitudinal axis, wherein the locking collar is configured to inhibit distal movement of the tether head assembly when the locking collar is in the locked configuration, and wherein the locking collar is configured to allow free movement of the tether head assembly along the longitudinal axis when the locking collar is in the unlocked configuration.

Example 24: the catheter of example 23, wherein the first locking element comprises a locking protrusion extending radially inwards from an inner surface of the locking collar, wherein the second locking element comprises a locking recess, and wherein when the locking collar is in the locked configuration, the locking protrusion is configured to be disposed within the locking recess of the tether head assembly to inhibit distal movement of the tether head assembly.

Example 25: the catheter of example 24, wherein the first locking element comprises a locking recess disposed within an inner surface of the locking collar, wherein the second locking element comprises a locking protrusion extending from the outer surface of the tether head assembly, and wherein when the locking collar is in the locked configuration, the locking recess is configured to receive the locking protrusion of the tether assembly to inhibit distal movement of the tether head assembly.

Example 26: the catheter of any of examples 22-25, wherein a proximal portion of the elongated body is removably secured to an elongated member coupled to the tether head assembly to inhibit unintended rotation of the tether head assembly.

Example 27: the catheter of any of examples 22-26, wherein the locking collar comprises an annular body defining an outer surface and an inner surface defining a locking collar lumen, wherein the locking collar lumen partially defines the catheter lumen, and wherein the first locking element is disposed on one or more of the outer surface or the inner surface of the locking collar.

Example 28: the catheter of example 27, wherein the first locking element is disposed on a proximal end of the locking collar towards the longitudinal axis of the catheter.

Example 29: the catheter of any of examples 22-28, wherein the first locking element comprises a same material as the locking collar.

Example 30: the catheter of any of examples 22-28, wherein the first locking element and the locking collar comprise different materials.

Example 31: a method comprising: advancing a tether head assembly of a tether assembly within a catheter lumen of an elongated body of a catheter to a locking collar disposed within a distal portion of the catheter via an elongated member coupled to a proximal end of the tether head assembly, wherein an inner surface of the locking collar defines a locking collar lumen in fluid communication with the catheter lumen; rotating the tether assembly about a longitudinal axis of the catheter from an unlocked orientation to a locked orientation to cause a first locking element of the locking collar to interface with a second locking element on the tether head assembly and inhibit unintended distal movement of the tether head assembly along the longitudinal axis; placing a medical device in an inner volume of a receptacle of the catheter, wherein the receptacle is affixed to the distal end of the elongated body; and attaching an attachment member of the medical device to the tether head assembly.

Example 32: the method of example 31, wherein the first locking element comprises a locking protrusion extending from the locking collar and towards the longitudinal axis, wherein the second locking element comprises a locking recess disposed on an outer surface of the tether head assembly, and wherein the locking protrusion is configured to be disposed within the locking recess when the tether assembly is in the locked orientation.

Example 33: the method of example 32, wherein the tether assembly comprises a projection extending from an outer surface of the tether head assembly, wherein the projection defines the locking recess.

Example 34: the method of example 31, wherein the first locking element comprises a locking recess disposed on an inner surface of the locking collar, wherein the second locking element comprises a locking protrusion extending radially outwards from an outer surface of the tether head assembly, and wherein the locking protrusion is configured to be disposed within the locking recess when the tether assembly is in the locked orientation.

Example 35: the method of any of claims 31-34, further comprising: securing the elongated member to a locking feature disposed along the elongated body of the catheter to inhibit unintended rotation of the tether assembly relative to the catheter.

Example 36: a method comprising: advancing a catheter through vasculature of a patient to a target tissue site, wherein the catheter comprises: an elongated body defining a catheter lumen, and a locking collar disposed within a distal portion of the catheter, the locking collar defining a first locking element, and a receptacle distal to the locking collar, wherein a tether head assembly of a tether assembly is disposed within the catheter lumen in a locked orientation, wherein the tether head assembly is coupled to an attachment member of a medical device, wherein the medical device is disposed within the receptacle without protruding from a distal end of the receptacle, and wherein a second locking element on an outer surface of the tether head assembly interfaces with the first locking element of the locking collar to inhibit unintended distal movement of the medical device; rotating the tether head assembly about a longitudinal axis of the catheter from the locked orientation to an unlocked orientation, wherein the second locking element does not interface with the first locking element when the tether head assembly is in the unlocked orientation; implanting the medical device within tissue at the target tissue site; and releasing the attachment member of the medical device from the tether head assembly.

Example 37: the method of example 36, wherein rotating the tether head assembly about the longitudinal axis from the locked orientation to the unlocked orientation comprises rotating an elongated coupled to the tether head assembly to cause the tether head assembly to rotate about the longitudinal axis, wherein implanting the medical device within the tissue at the target tissue site comprises rotating the medical device about the longitudinal axis to cause an elongated member defining a helix extending from distal end of the medical device to puncture the tissue.

Example 38: the method of example 37, wherein rotating the tether head assembly about the longitudinal axis from the unlocked orientation to the locked orientation comprises rotating the tether head assembly about the longitudinal axis in a first direction, wherein rotating the tether head assembly about the longitudinal axis from the locked orientation to the unlocked orientation comprises rotating the tether head assembly about the longitudinal axis in a second direction, and wherein the first direction is different from the second direction.

Example 39: the method of example 38, wherein implanting the medical device within the tissue comprises: rotating the tether head assembly about the longitudinal axis in a third direction to cause a distal tip of the elongated member of the medical device to puncture the tissue.

Example 40: the method of example 39, wherein the third direction is in a same direction as the second direction.

Example 41: the method of example 39, wherein the third direction is in a different direction from the first direction and is in a same direction as the first direction.

Example 42: the method of any of examples 39-41, further comprising: extending the medical device from a distal opening of the receptacle of the catheter via the tether head assembly; placing the distal tip of the elongated member of the medical device in contact with the tissue; and rotating the medical device about the longitudinal axis in the third direction by rotating the tether head assembly about the longitudinal axis in the third direction.

Example 43: the method of any of examples 36-42, wherein the first locking element comprises a locking protrusion extending from the locking collar and towards the longitudinal axis, wherein the second locking element comprises a locking recess disposed on an outer surface of the tether head assembly, and wherein the locking protrusion is configured to be disposed within the locking recess when the tether head assembly is in the locked orientation.

Example 44: the method of example 43, wherein the tether head assembly comprises a projection extending from an outer surface of the tether head assembly, wherein the projection defines the locking recess.

Example 45: the method of any of examples 36-42, wherein the first locking element comprises a locking recess disposed on an inner surface of the locking collar, wherein the second locking element comprises a locking protrusion extending radially outwards from an outer surface of the tether assembly, and wherein the locking protrusion is configured to be disposed within the locking recess when the tether head assembly is in the locked orientation.

Various aspects of the disclosure have been described. These and other aspects are within the scope of the following claims.

1. A system comprising:
a delivery catheter comprising:
an elongated body extending from a proximal end to a distal end along a longitudinal axis, wherein the elongated body defines a catheter lumen extending from the proximal end to the distal end;
a receptacle extending distally from the distal end of the elongated body along the longitudinal axis, the receptable defining an inner volume configured to retain an implantable medical device, wherein the inner volume is in fluid communication with the catheter lumen; and
a locking collar disposed at the distal end of the elongated body, the locking collar comprising a first locking element; and
a tether assembly configured to be disposed within the catheter lumen, wherein the tether assembly comprises:
a tether head assembly configured to receive an attachment member of the implantable medical device; and
a second locking element disposed on an outer surface of the tether head assembly, wherein the second locking element is configured to interface with the first locking element of the locking collar to inhibit unintended distal movement of the implantable medical device within the receptacle.

2. The system of claim 1, wherein the tether assembly is configured to transition between a locked orientation and an unlocked orientation relative to the locking collar via rotation of the tether assembly about the longitudinal axis, wherein the locking collar is configured to inhibit distal movement of the tether head assembly when the tether assembly is in the locked orientation, and wherein the locking collar is configured to permit distal movement of the tether head assembly when the tether assembly is in the unlocked orientation.

3. The system of claim 2, wherein when the tether assembly is in the locked orientation relative to the locking collar, the first locking element of the locking collar interfaces with the second locking element of the tether assembly.

4. The system of claim 2, wherein when the tether assembly is in the unlocked orientation relative to the locking collar, the first locking element of the locking collar does not interface with the second locking element of the tether assembly.

5. The system of claim 2,
wherein the first locking element comprises a locking protrusion extending radially inwards from the locking collar and towards the longitudinal axis,
wherein the second locking element comprises a locking recess on the outer surface of the tether head assembly, and
wherein when the tether assembly is in the locked orientation relative to the locking collar, the locking protrusion is disposed within the locking recess and interfaces with a sidewall of the locking recess to inhibit unintended distal movement of the implantable medical device within the receptacle.

6. The system of claim 5, wherein the retainer comprises a protrusion extending radially outwards away from the outer surface of the tether head assembly, and wherein the protrusion defines the locking recess.

7. The system of claim 2,
wherein the first locking element comprises a locking recess disposed on an inner surface of the locking collar,
wherein the second locking element comprises a locking protrusion extending radially outwards away from the longitudinal axis and the outer surface of the tether head assembly, and
wherein when the tether assembly is in the locked orientation relative to the locking collar, the locking protrusion is disposed within the locking recess and interfaces with a sidewall of the locking recess to inhibit unintended distal movement of the implantable medical device within the receptacle.

8. The system of claim 2, wherein the tether assembly is configured to affix the implantable medical device within tissue of a patient via the rotation of the tether assembly about the longitudinal axis, and wherein the tether assembly is configured to transition from the locked orientation to the unlocked orientation as a part of affixing the implantable medical device within the tissue.

9. The system of claim 8, wherein the implantable medical device defines an elongated body defining a helix extending from a distal end of the implantable medical device, wherein the helix is wound in a first direction, and wherein the tether assembly is configured to rotate about the longitudinal axis in the first direction to transition from the locked orientation to the unlocked orientation.

10. The system of claim 2, wherein the rotation of the tether assembly within the delivery catheter comprises a first rotation of the tether assembly, wherein the tether assembly is configured to affix the implantable medical device within tissue of a patient via a second rotation of the tether assembly about the longitudinal axis, wherein the first rotation is separate from the second rotation.

11. The system of claim 1, wherein the tether assembly comprises an elongated member coupled to a proximal end of the tether head assembly, wherein the elongated member is disposed within the catheter lumen, and wherein the elongated member is configured to be affixed to a proximal portion of the elongated body of the delivery catheter via a locking feature disposed along the proximal portion of the elongated body.

12. A tether head assembly comprising:
an inner retainer coupled and extending distally from a distal end of an elongated member;
an outer retainer defining a channel configured to receive the inner retainer, wherein the inner retainer and the outer retainer define a receptacle configured to receive an attachment member of a medical device;
a sheath configured to receive the inner retainer and the outer retainer; and
a first locking element disposed on an outer surface of the sheath,
wherein the tether head assembly is configured to be disposed within a catheter lumen of an elongated body of a delivery catheter, wherein the elongated member is configured to transmit a force from a proximal end of the elongated member to the inner retainer to move the tether head assembly within the catheter lumen of the delivery catheter, and wherein the first locking element is configured to interface with a second locking element disposed within an inner lumen to inhibit unintended movement of the tether head assembly towards a distal end of the elongated body of the delivery catheter.

13. The tether head assembly of claim 12,
wherein the force comprises a rotational force, and wherein the tether head assembly is configured to rotate about a longitudinal axis of the tether head assembly in response to the rotational force,
wherein the tether head assembly is configured to transition between a locked orientation and an unlocked orientation relative to the delivery catheter as a result of the rotation of the tether head assembly about the longitudinal axis,
wherein the first locking element of the tether head assembly is configured to interface with the second locking element of the delivery catheter when the tether head assembly is in the locked orientation, and
wherein the first locking element of the tether head assembly is configured to not interface with the second locking element of the delivery catheter when the tether head assembly is in the unlocked orientation.

14. The tether head assembly of claim 13, wherein the tether head assembly is configured to rotate about the longitudinal axis in a first direction about the longitudinal axis to affix the medical device within tissue of a patient, wherein the tether head assembly is configured to rotate about the longitudinal axis in a second direction about the longitudinal axis to transition from the locked orientation to the unlocked orientation.

15. The tether head assembly of claim 13, wherein the first locking element comprises a locking recess on the outer surface of the sheath, wherein the second locking element comprises a locking protrusion extending from an inner surface of the elongated body defining the catheter lumen and towards the longitudinal axis, and wherein locking recess is configured to receive the locking protrusion when the tether assembly is in the locked orientation.

16. The tether head assembly of claim 13, wherein the first locking element comprises a protrusion extending radially away from the outer surface of the sheath, wherein the second locking element comprises a locking recess dispose on an inner surface of the elongated body defining the catheter lumen, and wherein the protrusion is configured to be disposed within the locking recess when the tether assembly is in the locked orientation.

17. A catheter comprising:
an elongated body extending from a proximal end to a distal end along a longitudinal axis, wherein the elongated body defines a catheter lumen extending from the proximal end to the distal end;
a receptacle extending distally from the distal end of the elongated body along the longitudinal axis, the receptable defining an inner volume configured to retain an implantable medical device, wherein the inner volume is in fluid communication with the catheter lumen; and
a locking collar disposed at the distal end of the elongated body, the locking collar comprising a first locking element, wherein the first locking element is configured to interface with a second locking element on an outer surface of a tether head assembly disposed within the catheter lumen and attached to the implantable medical device to prevent unintended distal movement of the implantable medical device along the longitudinal axis.

18. The catheter of claim 17, where the locking collar is configured to transition between a locked configuration and an unlocked configuration in response to rotation of the tether head assembly about the longitudinal axis, wherein the locking collar is configured to inhibit distal movement of the tether head assembly when the locking collar is in the locked configuration, and wherein the locking collar is configured to allow free movement of the tether head assembly along the longitudinal axis when the locking collar is in the unlocked configuration.

19. The catheter of claim 18, wherein the first locking element comprises a locking protrusion extending radially inwards from an inner surface of the locking collar, wherein the second locking element comprises a locking recess, and wherein when the locking collar is in the locked configuration, the locking protrusion is configured to be disposed within the locking recess of the tether head assembly to inhibit distal movement of the tether head assembly.

20. The catheter of clam 17, wherein the locking collar comprises an annular body defining an outer surface and an inner surface defining a locking collar lumen, wherein the locking collar lumen partially defines the catheter lumen, and wherein the first locking element is disposed on one or more of the outer surface or the inner surface of the locking collar.
